SEQUENCE LISTING (16 sequences)

SEQ ID NO: 1 - protein, 20 aa, Homo sapiens
EDIIRNIARHLAQVGDSMDR

SEQ ID NO: 2 - protein, 20 aa, Homo sapiens
MRPEIWIAQELRRIGDEFNA

SEQ ID NO: 3 - protein, 20 aa, Homo sapiens [sequence omitted]

SEQ ID NO: 4 - protein, 24 aa, Homo sapiens
NLWAAQRYGRELRRMSDEFVDSFK

SEQ ID NO: 5 - protein, 20 aa, Homo sapiens
MEGSDALALRLACIGDEMDV

SEQ ID NO: 6 - protein, 20 aa, Homo sapiens
AELPPEFAAQLRKIGDKVYC

SEQ ID NO: 7 - protein, 20 aa, Homo sapiens
PADLKDECAQLRRIGDKVNL

SEQ ID NO: 8 - protein, 20 aa, Homo sapiens
SSAAQLTAARLKALGDELHQ

SEQ ID NO: 9 - protein, 20 aa, Homo sapiens
EQWAREIGAQLRRMADDLNA

SEQ ID NO: 10 - protein, 20 aa, Homo sapiens
HQAEVQIARKLQLIADQFHR

SEQ ID NO: 11 - protein, 20 aa, Homo sapiens
VVEGEKEVEALKKSADWVSD

SEQ ID NO: 12 - protein, 25 aa, Homo sapiens
NLWAAQRYGRELRRMSDEFVDSFKK

SEQ ID NO: 13 - protein, 25 aa, Homo sapiens
LWAAQRYGREARRMSDEFEGSFKGL

SEQ ID NO: 14 - protein, 23 aa, Homo sapiens
RPEIWMTQGLRRLGDEINAYYAR

SEQ ID NO: 15 - protein, 23 aa, Homo sapiens
RPEIWLTQSLQRLGDEINAYYAR

SEQ ID NO: 16 - protein, 23 aa, Homo sapiens
RREIWLTQHLQRLGDEINAYYAR

(19) United States
(12) Patent Application Publication
(10) Pub. No.: US 2018/0100859 A1
(43) Pub. Date: Apr. 12, 2018

(54) SURROGATE FUNCTIONAL BIOMARKER FOR SOLID TUMOR CANCER

(71) Applicant: EUTROPICS PHARMACEUTICALS, INC., Cambridge, MA (US)

(72) Inventors: Michael H. CARDONE, Dorchester, MA (US); Elisha J. DETTMAN, Dorchester, MA (US)

(21) Appl. No.: 15/560,636

(22) PCT Filed: Mar. 24, 2016
(86) PCT No.: PCT/US2016/023896

§ 371 Date: Sep. 22, 2017

Related U.S. Application Data
(60) Provisional application No. 62/137,591, filed on Mar. 24, 2015.

(51) Int. Cl. G01N 33/574; G01N 33/50; G01N 33/15

(52) U.S. Cl. CPC G01N 33/57484; G01N 33/5017; G01N 2800/7028; G01N 2800/52; G01N 33/15

(57) ABSTRACT

The present invention relates to diagnostic methods that are relevant to various solid tumor cancers that are not amenable to traditional BIH3 profiling diagnostic methods. In some embodiments, the methods described herein are useful in the evaluation of a patient, for example, for evaluating diagnosis, prognosis, and response to treatment. In various aspects, the present disclosure includes evaluating a solid tumor or cancer. In various embodiments, the evaluation may be selected from diagnosis, prognosis, and response to treatment. In various embodiments, the present disclosure directs the treatment of a cancer patient, including, for example, what type of treatment should be administered or withheld. In some embodiments, the present disclosure includes the measurement of a tumor cell, sample, and/or specimen, including biopsy or surgical specimen samples.

### CROSS-REFERENCE TO RELATED APPLICATIONS

This application claims priority to U.S. provisional application No. 62/137,591 filed Mar. 24, 2015, the contents of which are incorporated herein for all purposes.

### DESCRIPTION OF THE TEXT FILE SUBMITTED ELECTRONICALLY

The contents of the text file submitted electronically herewith are incorporated herein by reference in their entirety: a computer readable format copy of the sequence listing (filename: EUTR_016_{—01}WO_SeqList_ST25.txt, date recorded: Mar. 24, 2016, file size 4 kilobytes).

### FIELD OF THE INVENTION

The present disclosure relates to methods that are useful in evaluating solid tumors in human samples.

### BACKGROUND

The use of predictive and prognostic biomarkers paired with targeted cancer therapies may hold the key to reducing drug development time, improving drug efficacy, and guiding clinical decision making. While there have been advances in cancer treatment, chemotherapy remains largely inefficient and ineffective. The generally poor performance of chemotherapy may be attributed to the selected treatment not being closely matched to the individual patient's disease. A personalized medicine approach that couples precision diagnostics with therapeutics, especially targeted therapeutics, is a highly promising method for enhancing the efficacy of current and future drugs. Biomarkers can facilitate the development and use of such targeted therapeutics as well as standard of care therapies. To date there are only a handful of biomarkers useful in clinical oncology practice, in part because perceived markers often are correlative, but not causal, to the drug mechanism. Even when the biomarker biology does line up with the pharmacology of the companion therapy, predicting how a drug will work in a patient remains a challenge.

### SUMMARY OF THE INVENTION

In some aspects, the present disclosure provides a functional biomarker for predicting the response of solid tumor cells to apoptosis-inducing treatments. The method involves ex vivo treatment of biopsied and cryopreserved tumor cells with reagents (e.g. BH3 peptides and/or BH3 mimetics) that detect the state of the mitochondrial apoptosis signaling machinery. If the cell's apoptotic machinery is in a certain state (e.g. apoptotic), application of certain reagents results in measurable mitochondrial outer membrane permeabilization (MOMP), a key component of apoptosis. The disclosed method uses alternative dyes that allow the measurement of this mitochondrial response after the tumor cells have been treated and fixed, thereby preserving the signal for analysis after the assay. After the cells are fixed, the signal is measured, and the results (e.g. signal intensity and/or area under the curve) are entered into an algorithm to determine the percent mitochondrial priming. Multivariate analysis, which may include the percent mitochondrial priming, the presence of a Bcl-2 heterodimer, and/or other clinical factors, is used to predict treatment efficacy and direct clinical decisions.

In some aspects, the present disclosure provides a method of determining the apoptotic state of a solid cancer cell and/or sample by assaying thin sections of the tumor sample with mitochondrial profiling, incubating the cells and/or sample with a fixable dye, fixing the cells and/or sample, and measuring the signal in the cells and/or sample. In particular aspects, the cells in the sample are not further separated into single cell populations.

In some aspects, the method disclosed herein uses an antibody that specifically binds to Bcl-2 heterodimers to measure the apoptotic state of the cells in a solid tumor sample to correlate patient response with treatment (see, for example U.S. Pat. No. 8,168,755). In some aspects, the combination of the mitochondrial profiling and the detection of Bcl-2 heterodimers increases the sensitivity and/or specificity of the assay.

In some aspects, the present disclosure provides a method for selecting a cancer treatment for a patient with a solid tumor, including: a) obtaining a cancer cell or specimen from the patient; b) determining a mitochondrial profile for the cancer cell or specimen; c) determining one or more clinical factors of the patient, and d) classifying the patient for likelihood of clinical response to one or more cancer treatments; wherein the one or more clinical factors are selected to increase specificity and/or sensitivity of the mitochondrial profile for association with clinical response.

In some embodiments, the solid tumor is selected from non-small lung cell carcinoma, ovarian cancer, melanoma, breast cancer, prostate cancer, lung cancer, pancreatic cancer, colon cancer, hepatic cancer, and brain cancer. In some embodiments, the cancer treatment is one or more of anti-cancer drugs, chemotherapy, surgery, adjuvant therapy, and neoadjuvant therapy. In some embodiments, the cancer treatment is one or more of a BH3 mimetic, Her 2 antibody, Gemtuzimab, cisplatinin, EGFR inhibitor, Trail-1 ligand, epigenetic modifying agent, topoisomerase inhibitor, cyclin-dependent kinase inhibitor, and kinesin-spindle protein stabilizing agent. In some embodiments, the cancer treatment is a platinum-based therapeutic. In other embodiments, the platinum-based therapeutic is one or more of carboplatin, cisplatin, and oxaliplatin. In some embodiments, the cancer treatment is cytarabine or a cytarabine-based chemotherapy. In some embodiments, the cancer treatment is a BH3 mimetic. In other embodiments, the BH3 mimetic binds to one or more of BCL2, BCLXL, and MCL1. In some embodiments, the cancer treatment is an inhibitor of MCL1.

In some aspects, the method includes permeabilizing the cancer cells, determining a change in mitochondrial membrane potential upon contacting the permeabilized cells with one or more BH3 domain peptides using a fixable mitochondrial membrane potential dependent and fixable dye, the fixing cells with an aldehyde based fixative; and correlating a shift in mitochondrial membrane potential with chemosensitivity of the cells to apoptosis-inducing chemotherapeutic agents.

In some embodiments, the mitochondrial profile includes use of one or more BH3 domain peptides selected from the group consisting of BIM, BIM2A, BAD, BID, EIRK, PUMA, NOXA, BMF, BIK, and PUMA2A. In some embodiments, the one or more BH3 domain peptides are used at a concentration of 0.1 μM to 200 μM.

In some embodiments, the mitochondrial profile includes a BH3 mimetic compound that binds to all or selected members of the anti-apoptosis Bcl-2 family proteins. In some embodiments, the BH3 mimetic is used at a concentration of 0.01 μM to 100 μM in permeabilized cells. In some embodiments, the BH3 mimetic is used at a concentration of 0.01 μM to 100 μM in intact cells.

In some aspects, the specimen tested is a biopsy from a frozen tumor tissue specimen that has been cryosectioned, treated with mitochondrial membrane potential perturbing reagents, and fixed. In some embodiments, the specimen is a cancer stem cell. In some embodiments, the specimen is derived from a biopsy of the solid tumor. In some embodiments, the specimen is derived from the biopsy of a colorectal, breast, prostate, lung, pancreatic, renal, or ovarian primary tumor. In some embodiments, the specimen is of epithelial origin. In other embodiments, the epithelial specimen is enriched by selection from a biopsy sample with an anti-epithelial cell adhesion molecule (EpCAM) or other epithelial cell binding antibody bound to solid matrix or bead. In some embodiments, the specimen is of mesenchymal origin. In other embodiments, the mesenchymal specimen is enriched by selection from a biopsy sample with a neural cell adhesion molecule (N-CAM) or neuropilin or other mesenchymal cell binding antibody bound to a solid matrix or bead.

In some embodiments, the clinical factor is one or more of age, cytogenetic status, performance, histological subclass, gender, and disease stage. In some embodiments, the solid tumor is breast cancer and/or non-small cell lung cancer and the clinical factor is an age profile and/or cytogenetic status.

In some embodiments, the method includes measurement of an additional biomarker selected from mutational status, single nucleotide polymorphisms, steady state protein levels, and dynamic protein levels. In other embodiments, the method includes predicting a clinical response in the patient. In some embodiments, the clinical response is at least about 1, about 2, about 3, or about 5 year progression/event-free survival.

In some aspects, the likelihood of clinical response is defined by the following equation:

$$\% Priming = [ 100 * ( \frac{DMSO AUC - {Peptide}_{1} AUC}{DMSO AUC - {CCCP}_{avg} AUC} ) ] {Peptide}_{1} + [ 100 * ( \frac{DMSO AUC - {Peptide}_{2} AUC}{DMSO AUC - {CCCP}_{avg} AUC} ) ] {Peptide}_{2} + … / ( n peptides )$$

in which the AUC comprises either area under the curve or signal intensity; the DMSO comprises the baseline negative control; and the CCCP (Carbonyl cyanide m-chlorophenyl hydrazone) comprises an effector of protein synthesis by serving as uncoupling agent of the proton gradient established during the normal activity of electron carriers in the electron transport chain in the mitochondria comprises the baseline positive control. In some embodiments, the area under the curve is established by homogenous time-resolved fluorescence (HTRF). In some embodiments, the time occurs over a window from between about 0 to about 300 min to about 0 to about 30 min. In some embodiments, the area under the curve is established by fluorescence activated cell sorting (FACS) by the median fluorescence intensity (MFI) statistic. In some embodiments, the area under the curve is established by LI-COR. In other embodiments, the area under the curve is established by microscopy readout. In some embodiments, the signal intensity is a single time point measurement that occurs between about 5 min and about 300 min. For an individual peptide, priming may be calculated as:

$$Percentage Priming = [ 1 - \frac{( Peptide - CCCP )}{( DMSO - CCCP )} ] \times 100.$$

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE FIGURES

FIG. 1A-B shows a scheme for measuring mitochondrial outer membrane permeabilization (MOMP) in solid tumors. A solid tumor is biopsied and the sample core is cryopreserved (1). Thin sections of the frozen core are cut and placed in multiwall plate coated with Matrix proteins (2). The sections are treated with BH3 peptides or BH3 mimetic compounds and incubated (3). The mitochondria are labeled with a fixable dye, and the samples are fixed with para-formaldehyde. The dye emission signal is read on a LI-COR imager to detect shifts in mitochondrial outer membrane permeabilization in response to the analytes (4). Panel B shows a representative analyte response profile (concentration optimized).

FIG. 2 shows the percent heterodimer specific signal in breast cancer cells fixed and stained with an antibody that specifically binds to Bcl-xL/Bim heterodimers.

FIG. 3 shows flow cytometry and ELISA of pro-apoptotic-anti-apoptotic heterodimer specific antibody (HSBXB) signal correlates with mitochondrial BH3 profiling readout in cell lines: (A) Cells (AHR, DHL6 and MOLM13) were incubated on ice for 3 hours, and then washed and incubated with HSBXB antibody or Bcl-xL antibody at 10 ug/ml for 20 minutes, washed and stained with secondary Alexa488-conjugated goat anti-mouse. Signals are corrected to IgG-2A isotype or secondary alone control. (B) Hrk-BH3 signal in mitochondrial profiling of three cell lines plotted against normalized HSBXB FACS signal. (C). Anti-Bcl-xL capture of Bcl-xL-Bim complex from RIPA lysed cells. Captured complex probed with HSBXB or Bcl-xL.

### DETAILED DESCRIPTION OF THE INVENTION

Cancer cells, without wishing to be bound by theory, exhibit abnormalities, such as DNA damage, genetic instability, abnormal growth factor signaling, and abnormal or missing matrix interactions, any of which should typically induce apoptosis through the intrinsic (mitochondrial) apoptosis pathway. However, rather than respond to these apoptotic signals, cancer cells develop blocks in apoptosis pathways and survive. These blocks make some cancer cells resistant to some therapies, and, surprisingly, make some cancer cells sensitive to other therapies. The concept of “oncogene addiction” describes the phenomena of the acquired dependence of cancer cells on, or addiction to, particular proteins for survival.

Essentially all effective cancer drugs induce apoptosis in target cancer cells. However, different cancer cells respond to apoptosis-inducing drugs in different manners. Whether a cell will undergo apoptosis is based on competing pro- and anti-apoptotic signaling converging at the mitochondrion. The decisive event that commits the cell to death via the intrinsic, or mitochondrial, pathway of apoptosis is mitochondrial outer membrane permeabilization (MOMP). In many cases, MOMP is the point of no return in the intrinsic apoptosis pathway.

MOMP is regulated through different cellular pathways, one of which involves the Bcl-2 family, a group of mitochondrial proteins known to play a key role in apoptosis. This family is composed of three groups: multidomain anti-apoptotic proteins (e.g. Bcl-2, Bcl-xL, Bfl-1, and Mcl-1), BH3-domain only pro-apoptotic proteins (e.g. Bid, Bim, Bad, Bik, Noxa, Hrk, and Puma), and multidomain pro-apoptotic proteins (e.g. Bax and Bak). Further, there are two groups of BH3-domain only proteins: the activators (e.g. Bim and Bid) and the sensitizers (e.g. Bad, Bik, Noxa, Hrk, Bmf, and Puma).

Bcl-2 protein activity is regulated by distinct interactions between pro-survival (anti-apoptotic) and pro-apoptotic family members which occur primarily through BH3 (Bcl-2 homology domain-3) mediated binding. Apoptosis-initiating signaling occurs for the most part upstream of the mitochondria, and causes the translocation of BH3-only proteins to the mitochondria where they either activate or sensitize MOMP. The activator BH3-only proteins bind to, and directly activate, pro-apoptotic proteins. These activators can also bind to and inhibit the anti-apoptotic Bcl-2 family proteins. This binding sequesters the activator proteins and prevents them from exerting their apoptotic activity. Displacement of the activators by sensitizer peptides results in Bax/Bak-mediated apoptotic commitment. The sensitizer BH3 proteins bind only to the anti-apoptotic Bcl-2 family proteins and block their anti-apoptotic functions. Without wishing to be bound by theory, each sensitizer protein has a unique specificity profile. For example, Noxa (A and B) bind with high affinity to Mcl-1, Bad binds to Bcl-xL and Bcl-2 but only weakly to Mcl-1, and Puma binds well to all three targets. These interactions can have various outcomes, including homeostasis, cell death, sensitization to apoptosis, and blockade of apoptosis.

A defining feature of cancer cells in which apoptotic signaling is blocked is an accumulation of sequestered BH3-only activator proteins at the mitochondrial surface. This accumulation brings the BH3-only activator proteins into proximity to their effector target proteins resulting in “mitochondrial priming”. Antagonism of anti-apoptotic family members in this primed state results in MOMP and apoptosis.

Different cancer cells are dependent on different apoptosis-regulating proteins for survival. Identifying which of these proteins are mediating cell survival in a given cancer cell provides insight into the likelihood of a cancer cell to respond to a particular treatment. In mitochondrial profiling, the mitochondria in a cell or sample are exposed to known concentrations of BH3 peptides. If the BH3 peptides tested disrupt a Bcl-2 heterodimer that sequestered a pro-apoptotic polypeptide, the now-freed polypeptide is activated, resulting in a measurable increase in MOMP.

Disclosed herein are methods for determining which Bcl-2 proteins play a role in determining the apoptotic state of a cancer. By simultaneously assaying the activity of multiple Bcl-2 proteins at the mitochondria, a mitochondrial profile is provided that gives a measure of each Bcl-2 protein's contribution to apoptosis resistance.

Mitochondrial profiles may be prepared as disclosed herein. The underlying molecular principle applies across all tissue types, however because the assay traditionally requires viable, dissociated cells, the format described in Application Number PCT/US2014/063121 for example is not amenable for use in solid tumors. The process of dissociating tumor cells from the tumor mass affects the functional biomarker, thereby limiting the use of mitochondrial profiling of those tumors. Sensitive and/or specific mitochondrial profiling measurements can be made on solid tumors if the signal can be fixed following application of heterodimer perturbing agents (e.g. BH3 peptides or BH3 mimetics)

The detection of the apoptotic state of the cancer allows analysis of the cancer's predicted response to treatment in the context of certain clinical factors and other measurements of the natural state of the cancer cell. The diagnostic approaches described herein allow for analysis of a suite of mitochondrial response profiles and clinical indicators, including ones not directly related to apoptosis, for predicting therapeutic efficacy in human malignancies. Combining measurements taken on the solid cancer cell with other clinical variables provides highly sensitive predictive tests for guiding treatment.

### BH3 Peptides and BH3 Mimetics

Mitochondrial profiling measures a cell's apoptotic state using a panel of BH3 peptides derived from BH3 domains of BH3-only proteins (e.g. those shown in Table 1), and/or small molecule mimetics of these peptides that selectively antagonize individual BCL-2 family members. The degree to which MOMP occurs in a cell after exposure to the panel of peptides indicates the cell's likelihood to undergo apoptosis in response to chemotherapy (see for example U.S. Pat. No. 8,221,966).

The BH3 panel may include peptides derived from, for example, BCL-2, BCL-XL, BCL-w, MCL-1 and BFL-1. Each of anti-apoptotic proteins BCL-2, BCL-XL, MCL-1, BFL-1 and BCL-w bear a unique pattern of interaction with this panel of proteins. Anti-apoptotic family members may be distinguished from each other based on their affinity for individual BH3 domains. For instance, BCL-XL may be distinguished from BCL-2 and BCL-w by its greater affinity for HRK BH3. In contrast MCL-1 does not bind BAD BH3 (Opferman et al. 2003). In some embodiments, the BH3 peptides are those described in or are derived from those described in Foight et al. ACS Chem. Biol. 2014, 9, 1962-1968.

TABLE 1
BH3 peptides

| BH3 | | SEQ |
|---|---|---|
| peptide | Amino Acid Sequence | ID NO |
| BID | EDIIRNIARHLAQVGDSMDR | 1 |
| BIM | MRPEIWIAQELRRIGDEFNA | 2 |
| BID mut | EDIIRNIARHAAQVGASMDR | 3 |
| BAD | NLWAAQRYGRELRRMSDEFVDSFK | 4 |
| BIK | MEGSDALALRLACIGDEMDV | 5 |
| NOXA A | AELPPEFAAQLRKIGDKVYC | 6 |
| NOXA B | PADLKDECAQLRRIGDKVNL | 7 |
| HRK | SSAAQLTAARLKALGDELHQ | 8 |
| PUMA | EQWAREIGAQLRRMADDLNA | 9 |
| BMF | HQAEVQIARKLQLIADQFHR | 10 |
| BNI | VVEGEKEVEALKKSADWVSD | 11 |
| huBAD | NLWAAQRYGRELRRMSDEFVDSFKK | 12 |
| BADmut | LWAAQRYGREARRMSDEFEGSFKGL | 13 |
| MS-1 | RPEIWMTQGLRRLGDEINAYYAR | 14 |
| MS-2 | RPEIWLTQSLQRLGDEINAYYAR | 15 |
| MS-3 | RREIWLTQHLQRLGDEINAYYAR | 16 |

The BH3 panel can include peptides of any length. In some embodiments, the peptide length is less than about 200 amino acids. In some embodiments, the peptide length is less than about 150 amino acids. In some embodiments, the peptide length is less than about 140, about 130, about 120, about 110, about 100, about 90, about 50, about 40, about 30, about 20, or about 10 amino acids.

The BH3 panel can further include variants of the BH3 domains or mimetics thereof. For example, a BH3 domain peptide can include a peptide which includes (in whole or in part) the sequence NH2-XXXXXXIAXXLXXXGDXXXX—COOH or NH2-XXXXXXXXXXLXXXXDXXXX—COOH. The BH3 domain can include at least about 5, about 6, about 7, about 8, about 9, about 10, about 15, or about 20 or more amino acids of any of SEQ ID NOs: 1-16. Preferred variants are those that have conservative amino acid substitutions made at one or more predicted non-essential amino acid residues. For example, a “conservative amino acid substitution” is one in which the amino acid residue is replaced with an amino acid residue having a similar side chain. In further embodiments, the BH3 domain peptide is an activator or a sensitizer of apoptosis. In preferred embodiments, the BH3 domain peptide is a sensitizer.

In various embodiments, the BH3 panel may include one or more BH3 mimetics. For example, a BH3 mimetic compound targeting Bcl-2 and Bcl-xL (e.g. Abt-263) or a BH3 mimetic compounds targeting Mcl-1 (e.g. EU-5148) may be used. BH3 mimetics or analogs thereof, that may be used in the present invention include, but are not limited to, Gossypol and its analogs (e.g. Ideker et al. Genome Res 2008), ABT-199, ABT-737 (e.g. Petros et al. Protein Sci. 2000), ABT-263 (e.g. Letai et al. Cancer Cell 2002) and their analogues (e.g. WO2005049593, U.S. Pat. No. 7,767,684, U.S. Pat. No. 7,906,505), Obatoclax (e.g. WO2004106328, WO2005117908, U.S. Pat. No. 7,425,553), EU-5148, EU-5346, EU-4030, EU-51aa48 (Eutropics), compounds that selectively inhibit Mcl-1 (e.g. WO2008131000, WO2008130970, Richard, et al. (2013) Bioorg Med Chem. 21(21):6642-9)), HA-14-1 (e.g. Wang, et al. (2000) Proc. Natl. Acad. Sci. USA 97: 7124-9), Antimycin-A (e.g. Tzung, et al. (2001) Nat. Cell. Biol. 3: 183-191), BH3I-1 and BH3I-2 (e.g. Degterev, et al. (2001) Nat. Cell. Biol. 3: 173-82), terphenyl derivatives (e.g. Kutzki, et al. (2002) J. Am. Chem. Soc. 124: 11838-9), and compounds with selective BH3 mimic function (e.g. Ng (2014) Clin Adv Hematol Oncol. 12(4):224-9. In some embodiments, the one or more BH3 mimetic compounds antagonize one, some, or all selected BH3 binding in the assay.

In some embodiments the BH3 panel may include use of a stapled peptide (e.g. a peptide generated through the synthetic enhancement of a 3-D alpha-helix protein segment with hydrocarbon bonds to make proteins more rigid and able to penetrate cells), as described in, for example, Verdine, et al. “Stapled Peptides for Intracellular Drug Targets” Methods in Enzymology, Volume 503 (Chap. 1).

### Solid Tumor Mitochondrial Profiling

In some embodiments, the assay is performed during the course of cancer treatment to identify patients that are likely to respond to a given treatment, who might relapse, or for whom treatment may otherwise lose efficacy. The assay can identify cancers that during treatment shift in their sensitivity to any class of drugs that directly or indirectly induce apoptosis through the mitochondrial apoptosis pathway.

Mitochondrial profiling includes associating the propensity of a pro-apoptotic peptide to induce mitochondrial depolarization (% priming) and patient classification (e.g. responder/non-responder). In other embodiments, the application of an algorithm to the percent priming by any particular BH3 peptide, mimetic, or combination thereof is associated with patient classification (e.g. responder/non-responder).

Mitochondrial profiling and reagents useful for such a method are described for example in U.S. Pat. Nos. 7,868,133; 8,221,966; and 8,168,755 and US Patent Publication No. 2011/0130309.

In some embodiments, the present methods provide a multi peptide analysis, as opposed to an evaluation of a single BTU peptide. In some embodiments, a panel of BH3 peptides is screened on a single patient specimen. For example, the disclosure provides mitochondrial profiling in which at least two, or three, or four, or five, or six, or seven, or eight, or nine, or ten BH3 peptides are evaluated at once.

In some embodiments, the BH3 peptide or BH3 mimetic is used at a concentration of about 0.1 to about 200 μM. In some embodiments, the BH3 peptide or BH3 mimetic is used at a concentration of about 0.1 to about 150, or about 0.1 to about 100, or about 0.1 to about 50, or about 0.1 to about 10, or about 0.1 to about 5, about 1 to about 150, or about 1 to about 100, about 1 to about 50, about 1 to about 10, about 1 to about 5 μM, or about 10 to about 100 μM of the peptide or mimetic is used. In some embodiments, the BH3 peptide or BH3 mimetic is used at a concentration of about 0.1, or about 0.5, or about 1.0, or about 5, or about 10, or about 50, or about 100, or about 150, or about 200 μM of the BH3 peptide or BH3 mimetic is used. In some embodiments, the solid tumor mitochondrial profiling includes permeabilizing the cancer cells, sample, and/or specimen. In other embodiments the solid tumor mitochondrial profiling includes adding the BH3 mimetic compound or BH3 peptide to the mitochondria without permeabilizing the outer membrane.

In various aspects, the disclosure provides methods of predicting sensitivity of a cell to a therapeutic agent by contacting the cell with a BH3 domain peptide and/or mimetic and detecting MOMP both before and after contacting said cell with a therapeutic agent. In some embodiments, the mitochondrial profiling includes performing a mitochondrial profile before cancer treatment begins, and then performing another mitochondrial profile during treatment. A shift of the mitochondrial profile in the cancer cell after treatment compared to the initial mitochondrial profile provides a pharmacodynamic marker to indicate the cancer cell's resistance or sensitivity and predict response to treatment. In some embodiments, the decision to perform subsequent mitochondrial profiling in a patient is made when the patient stops responding to a current course of treatment. In other embodiments, the decision to perform subsequent mitochondrial profiling is made independently of the patient's response to treatment.

In some embodiments, the mitochondrial profiling includes subjecting a patient cancer cell or specimen to a BH3 panel, and comparing the mitochondrial profile of the patient sample to that of a test cell or specimen (e.g. from an individual without cancer, a nave patient, or the same patient before treatment). The method may further include comparing the BH3 panel read-out between the patient or test sample, and correlating any differences in the mitochondrial profile of the sample to sensitivity and/or resistance to a particular treatment. In further embodiments, an algorithm is applied to the read-outs between the patient and test samples and the results of the algorithm are correlated with any differences in sample sensitivity and/or resistance to a particular treatment.

In some embodiments, the information from the mitochondrial profile is considered in conjunction with the detection of Bcl-2 heterodimers formed in the cancer cells, sample, and/or specimen. In some embodiments, the presence of a Bcl-2 heterodimer is detected using an antibody directed against one of more of BIM, BIM2A, BAD, BID, HICK, PUMA, NOXA, BMF, BIK, PUMA2A, and naturally-occurring heterodimers formed between two Bcl-2 proteins, e.g. a first Bcl-2 protein (e.g., Bim, Bid, Bad, Puma, Noxa, Bak, Hrk, Bax, or Mule) and a second Bcl-2 protein (e.g., Mcl-1, Bcl-2, Bcl-XL, Bfl-1 or Bcl-w) as described in U.S. Pat. No. 8,168,755. In some embodiments, the antibody detects a Bcl-xL/Bim heterodimer.

In other aspects, the disclosure provides a method for determining a mitochondrial profile for a patient's solid tumor cell specimen; determining one or more clinical factors of the patient where the one or more clinical factors are selected from age profile and/or cytogenetic status; and classifying the patient for likelihood of clinical response to one or more cancer treatments.

The mitochondrial profiling may be performed on permeabilized or non-permeabilized cells. In some embodiments, the mitochondrial profiling includes permeabilizing a patient's cancer cells, determining or quantifying a change in mitochondrial membrane potential upon contacting the permeabilized cells with one or more BH3 domain peptides and/or BH3 mimetics for a time determined to allow membrane depolarization, and applying a fixative to stabilize the potentiometric dye signal. Measurements are taken for signal intensity for each peptide, and the readouts are combined with the clinical factors or measurements described herein and are used to differentiate patient response and/or patients for a variety of therapies. In some embodiments, the mitochondrial profiling exposes a patient's cancer cells to BH3 mimetic compounds that antagonize all or selected BH3 mediated binding and determining or quantifying a change in mitochondrial membrane potential upon contacting the permeabilized or intact cells.

In various aspects, the disclosure provides applying an algorithm to the results of the mitochondrial profiling, and analyzing the pattern and/or degree of response in the mitochondrial profile to predict the cell or specimen sensitivity to treatment. In some embodiments, sequential biomarker algorithms derived from assessment of the mitochondrial profile are applied to classify a patient according to likely response to treatment. In some embodiments, the algorithm is applied to predict the shift in cell response (e.g. sensitivity or resistance) as measured in the mitochondrial profile. In a non-limiting example, BIM and NOXA metrics are critical determinants of 5-Azacitidine response. (See Bogenberger et al. Leukemia (2014)).

In certain embodiments, the likelihood of response is determined by assessing percent mitochondrial priming. In certain embodiments, the percent mitochondrial priming is defined by the following equation:

$$\% Priming = [ 100 * ( \frac{DMSO AUC - {Peptide}_{1} AUC}{DMSO AUC - {CCCP}_{avg} AUC} ) ] {Peptide}_{1} + [ 100 * ( \frac{DMSO AUC - {Peptide}_{2} AUC}{DMSO AUC - {CCCP}_{avg} AUC} ) ] {Peptide}_{2} + … / ( n peptides )$$

in which the AUC comprises either area under the curve or signal intensity; the DMSO comprises the baseline negative control; and the CCU) (Carbonyl cyanide m-chlorophenyl hydrazone) comprises an effector of protein synthesis by serving as uncoupling agent of the proton gradient established during the normal activity of electron carriers in the electron transport chain in the mitochondria comprises the baseline positive control. In some embodiments, the area under the curve is established by homogenous time-resolved fluorescence (HTRF). In some embodiments, the time occurs over a window from between about 0 to about 300 min to about 0 to about 30 min. In some embodiments, the area under the curve is established by fluorescence activated cell sorting (FACS) by the median fluorescence intensity (MFI) statistic. In some embodiments, the area under the curve is established by LI-COR IR imaging system which uses near-infrared (IR) fluorophores (670-1100 nm) that have a distinct advantage over visible dyes, provides quantitative signal analysis in fixed cells and solid tumor thin sections. In some embodiments, the signal is detected by LI-COR imaging.

In some embodiments, the signal intensity is a single time point measurement that occurs between about 5 min and about 300 min. For an individual peptide, priming may be calculated as:

$$Percentage Priming = [ 1 - \frac{( Peptide - CCCP )}{( DMSO - CCCP )} ] \times 100.$$

In other embodiments the mitochondrial membrane potential shift is measured by Immunofluorescence (IF) microscopy. This is carried out using an imaging system and microscope. In some embodiments, the signal intensity is a single time point measurement that occurs between about 5 min and about 300 min.

In other embodiments the primed state is determined by measuring the occurrence of heterodimers of pro-and anti-apoptotic proteins using enzyme linked immunosorbent assay (ELISA). This can be accomplished in solid tumor tissue using a heterodimer specific antibody that recognizes presence of the pro-apoptotic and anti-apoptotic proteins in complex. (U.S. Pat. No. 8,168,755 “Antibodies specific to heterodimers of Bcl-2 family and uses thereof.”)

In another embodiment the primed state of the cancer cell is determined by directly measuring the occurrence of heterodimers of pro-and anti-apoptotic proteins in fresh or fresh frozen biopsied specimens taken from the tumor or from the circulating tumor cells from cancer patients. In one case the occurrence of heterodimers of pro-and anti-apoptotic proteins is determined using heterodimer specific antibodies or conventional antibodies against the anti-apoptotic partner of the heterodimer complex in an ELISA format. The extent of priming is determined as the ratio of the signal achieved using the heterodimer specific antibody to the signal achieved using the non-selective antibody for the anti-apoptotic member of the complex. To better enable the detection of the complex and to minimize specimen sample requirement the ELISA can be performed on a micro-bead surface and detected using a highly sensitive digital ELISA in microfluidic format called Simoa™ from Quanterix Inc. (Lexington, Mass.). The unusually high sensitivity of the assay enables the use of ELISA in detecting quantitation of as little as a single molecule and enables assessment of fine needle aspirate as well as liquid biopsies from cancer patients.

In another embodiment the primed state of a circulating cancer cell is determined by directly measuring the occurrence of heterodimers of pro-and anti-apoptotic proteins in fresh serum or blood samples using Flow cytometry. In this case the circulating cancer cell is identified using antibodies to cell surface markers such as EpCam (Proc. Natl. Acad. Sci. USA. 106: 3970-3975, doi: 10.1073/pnas.0813188106) or cytokeratines (CKs) (Transl Lung Cancer Res. 2013 April; 2(2): 65-71). These cells are simultaneously stained for the heterodimers of pro- and anti-apoptotic specific signal and normalized to the expression level of the entire anti-apoptotic protein, for example; (Bcl-xL-Bim)/homodimer signal (Bcl-xL)

### Clinical Decisions

In some embodiments, the methods described herein are useful in the evaluation of a patient, for example, for evaluating diagnosis, prognosis, and response to treatment. In various aspects, the present disclosure includes evaluating a solid tumor or cancer. In various embodiments, the evaluation may be selected from diagnosis, prognosis, and response to treatment.

Diagnosis refers to the process of attempting to determine or identify a possible disease or disorder, such as, for example, cancer. Prognosis refers to predicting a likely outcome of a disease or disorder, such as, for example, cancer. A complete prognosis often includes the expected duration, the function, and a description of the course of the disease, such as progressive decline, intermittent crisis, or sudden, unpredictable crisis. Response to treatment is a prediction of a patient's medical outcome when receiving a treatment. Responses to treatment can be, by way of non-limiting example, pathological complete response, survival, progression free survival, time to progression, and probability of recurrence.

In various embodiments, the present methods direct a clinical decision regarding whether a patient is to receive a specific treatment. In some embodiments, the present methods are predictive of a positive response to neoadjuvant and/or adjuvant chemotherapy, or a non-responsiveness to neoadjuvant and/or adjuvant chemotherapy. In some embodiments, the present methods are predictive of a positive response to a pro-apoptotic agent or an agent that operates via apoptosis and/or an agent that does not operate via apoptosis or a non-responsiveness to apoptotic effector agent and/or an agent that does not operate via apoptosis. In various embodiments, the present disclosure directs the treatment of a cancer patient, including, for example, what type of treatment should be administered or withheld.

In some embodiments, a comparison of the data generated in the mitochondrial profile performed at various time points during treatment shows a change in profile readout indicating a change in the cancer's sensitivity to a particular treatment. In some embodiments, the determination of a cancer's change in sensitivity to a particular treatment is used to re-classify the patient and to guide the course of future treatment.

In some embodiments, the determination of the sensitivity or resistance of a patient's cancer cell to a particular therapeutic is used to classify the patient into a treatment or prognosis group. In some non-limiting examples, patients are classified into groups designated as cure, relapse, no complete response, complete response, refractory to initial therapy, responder, non-responder, high likelihood of response, or low likelihood of response. In further embodiments, analysis of the mitochondrial profiling and patient classification direct a clinical decision regarding treatment, such as, for example, switching from one therapeutic to another, a change in dose of therapeutic, or administration of a different type of treatment (e.g. surgery, radiation, allogenic bone marrow or stem cell transplant). In a further embodiment, clinical decision is directed by the analysis of a change in cancer sensitivity, classification, and consideration of clinical factors, such as age and/or cytogenetic status. In various embodiments, a cancer treatment is administered or withheld based on the methods described herein. Exemplary treatments include surgical resection, radiation therapy (including the use of the compounds as described herein as, or in combination with, radiosensitizing agents), chemotherapy, pharmacodynamic therapy, targeted therapy, immunotherapy, and supportive therapy (e.g., painkillers, diuretics, antidiuretics, antivirals, antibiotics, nutritional supplements, anemia therapeutics, blood clotting therapeutics, bone therapeutics, and psychiatric and psychological therapeutics).

In some embodiments, the methods described herein provide a diagnostic test that is predictive of a response treatment for cancer patients matching a cytogenetic profile or status and/or is of a certain age. In some embodiments, the diagnostic test measuring membrane potential in solid tumors includes measuring a change in mitochondrial membrane potential in response to BH3 mimetic compounds and/or BH3 containing peptides. In some embodiments, the cancer is breast cancer. In some embodiments, the cancer treatment is HERCEPTIN™ (trastuzumab) treatment.

In other aspects, the disclosure provides a method for determining a cancer patient response to one or more Bcl-2 proteins and/or BH3 mimetic-targeted treatments (e.g. targeting Bcl-2, Mcl-1, or Bcl-xL); determining one or more Bcl-2 protein dependencies of the patient, and classifying the patient for likelihood of clinical response to one or more BH3 mimetic compounds or other Bcl-2 family protein perturbing treatments.

Cancer cells identified through mitochondrial profiling as dependent on particular members of the Bcl-2 family to survive are expected to be sensitive to particular therapies. For example, a cell yielding a high signal, relative to the positive control, of Noxa (A or B) peptide-induced MOMP is Mcl-1 primed (e.g. Mcl-1 provides the apoptotic block and displacement of this polypeptide will allow apoptosis to proceed), while a high response to the peptide Bad indicates that Bcl-xL or Bcl-2 provides the apoptotic block. In some embodiments, a cell yielding a high apoptotic response to Puma reflects pan-Bcl-2 family priming. In this way, cells that are dependent on either Mcl-1 or Bcl-xL, on both proteins, or on several Bcl-2 family members are readily distinguished so that appropriate treatment may be tailored accordingly. The distinctions in mitochondrial response to these peptides guides the use of therapies that are known to work through pathways that funnel into either Mcl-1 or Bcl-xL affected intrinsic signaling. The use of a Bcl-2-inhibiting or a Mcl-1-inhibiting compound may be indicated in such cases. In some embodiments, the present methods also indicate or contraindicate therapies that target entities upstream of Mcl-1 or Bcl-xL. For example, cancer cells that are dependent on the Bcl-2 protein, but not the Mcl-1 protein, will be responsive to a drug that specifically targets that protein, such as the Abbott ABT-199 drug. The sensitivity of the cancer to a particular therapeutic can be monitored during treatment by performing the mitochondrial profile at various time points during the course of treatment. If, for example, the mitochondrial profile shifts during the course of treatment to indicate sensitivity to a different BH3 peptide, e.g. a Bcl-xl dependence, then the treatment would be changed to a drug that targets Bcl-xl, e.g. Abbott ABT-263 drug. If, for example, the profile shift indicates a dependence on the Mcl-1 protein, as indicated by response to the NOXA peptide, a drug that targets Mcl-1, e.g. Eutropics EU-5148 (E), would be appropriate. This information will guide the use of the appropriate drugs that have an apoptosis independent mechanism of action in conferring cytotoxicity through perturbation of metabolic pathways such as electron transport inhibitors (e.g. rotenone), uncoupling reagents (e.g. dinitrophenol) or oxidative phosphorylation inhibitors (e.g. oligomycin).

In some embodiments, the disclosure predicts the efficacy of a cancer treatment which can include one or more of anti-cancer drugs, chemotherapy, surgery, adjuvant therapy (e.g. prior to surgery), and neoadjuvant therapy (e.g. after surgery). In an exemplary embodiment, the present method indicates whether a patient is to receive a pro-apoptotic agent or an agent that operates via apoptosis for cancer treatment. In another exemplary embodiment, the present method indicates whether a patient is to receive an agent that does not operate via apoptosis.

In some embodiments, the likelihood of response to a particular treatment is determined through multivariate analysis. In some embodiments, the multivariate analysis includes one or more of the percent mitochondrial priming, the presence or absences of one or more Bcl-2 heterodimers in the same patient and/or tumor, and/or other clinical factors.

In some embodiments, the present methods direct a clinical decision regarding whether a patient is to receive adjuvant therapy after primary, main or initial treatment, including, without limitation, a single sole adjuvant therapy. Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease.

In some embodiments, the present methods direct a patient's treatment to include adjuvant therapy. For example, a patient that is scored to be responsive to a specific treatment may receive such treatment as adjuvant therapy. Further, the present methods may direct the identity of an adjuvant therapy, by way of non-limiting example, as a treatment that induces and/or operates in a pro-apoptotic manner or one that does not. In some embodiments, the present methods may indicate that a patient will not be or will he less responsive to a specific treatment and therefore such a patient may not receive such treatment as adjuvant therapy. Accordingly, in some embodiments, the present methods provide for providing or withholding adjuvant therapy according to a patient's likely response. In this way, a patient's quality of life, and the cost of care, may he improved.

In various embodiments, the present methods direct a clinical decision regarding whether a patient is to receive neoadjuvant therapy, e.g. therapy to shrink and/or downgrade the tumor prior to surgery. In some embodiments, neoadjuvant therapy means chemotherapy administered to cancer patients prior to surgery. In some embodiments, neoadjuvant therapy means an agent, including those described herein, administered to cancer patients prior to surgery. Types of cancers for which neoadjuvant chemotherapy is commonly considered include, for example, breast, colorectal, ovarian, cervical, bladder, and lung.

In some embodiments, the present methods direct a patient's treatment to include neoadjuvant therapy. For example, a patient that is scored to be responsive to a specific treatment may receive such treatment as neoadjuvant therapy. Further, the present methods may direct the identity of a neoadjuvant therapy, by way of non-limiting example, as a treatment that induces and/or operates in a pro-apoptotic manner or one that does not. In some embodiments, the present methods may indicate that a patient will not be or will be less responsive to a specific treatment and therefore such a patient may not receive such treatment as neoadjuvant therapy. Accordingly, in some embodiments, the present methods provide for providing or withholding neoadjuvant therapy according to a patient's likely response. In this way, a patient's quality of life, and the cost of case, may be improved.

In some embodiments, the present methods provide a high likelihood of response to a particular treatment and may direct treatment, including aggressive treatment. In some embodiments, the present methods provide a low likelihood of response to a particular treatment and may direct cessation of treatment, including aggressive treatment, and the use of palliative care, to avoid unnecessary toxicity from ineffective chemotherapies for a better quality of life.

In some embodiments, the methods disclosed herein include preventative measures such as administering a treatment to a patient that is likely to be afflicted by cancer as guided by the methods described herein.

### Treatments

In exemplary embodiments, the disclosure selects a treatment. Exemplary treatments include surgical resection, radiation therapy (including the use of the compounds as described herein as, or in combination with, ra.diosensitizing agents), chemotherapy, pharmacodynamic therapy, targeted therapy, immunotherapy, and supportive therapy (e.g., painkillers, diuretics, antidiuretics, antivirals, antibiotics, nutritional supplements, anemia therapeutics, blood clotting therapeutics, bone therapeutics, and psychiatric and psychological therapeutics). In some embodiments, the cancer treatment is one or more of a BH3 mimetic (by way of non-limiting example, one or more of BCL2, BCLXL, MCL1, Abt-263, EU-51aa48, EU-5346, and EU-5148);), epigenetic modifying agent, topoisomerase inhibitor, cyclin-dependent kinase inhibitor, EGFR antagonist, and/or kinesin-spindle protein stabilizing agent. In still other embodiments, the cancer treatment includes one or more of a proteasome inhibitor; a modulator of cell cycle regulation (by way of non-limiting example, a cyclin dependent kinase inhibitor); a modulator of cellular epigenetic mechanistic (by way of non-limiting example, one or more of a histone deacetylase (HDAC) (e.g. one or more of vorinostat or entinostat), azacytidine, decitabine); an anthracycline or anthracenedione (by way of non-limiting example, one or more of epirubicin, doxorubicin, mitoxantrone, daunorubicin, idarubicin); a platinum-based therapeutic (by way of non-limiting example, one or more of carboplatin, cisplatin, and oxaliplatin); cytarabine or a cytarabine-based chemotherapy; and/or an inhibitor of MCL1.

In some embodiments, the cancer treatment is a pro-apoptotic agent. In various embodiments, pro-apoptotic agents and/or agents that operate via apoptosis and/or an agent that operates via apoptosis driven by direct protein modulation include, but are not limited to, ABT-263 (Navitoclax), and obatoclax, WEP, bortezomib, and carfilzomib.

In some embodiments, the cancer treatment is an agent that does not operate via apoptosis. Agents that do not operate via apoptosis include, but are not limited to, kinesin spindle protein inhibitors, cyclin-dependent kinase inhibitor, Arsenic Trioxide (TRISENOX), MEK inhibitors, pomolidomide, azacytidine, decitibine, vorinostat, entinostat, dinaciclib, antibody-drug conjugates (e.g. gemtuzumab), BTK inhibitors, PI3 kinase delta inhibitors, lenolidimide, anthracyclines, cytarabine, melphalam, Akt inhibitors, mTOR inhibitors.

In various embodiments, the disclosure pertains to cancer treatments including, without limitation, one or more of alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR inhibitor (e.g., erlotinib (Tarceva)) and VEGF-A that reduce cell proliferation, dacogen, a HER2 antibody (e.g. trastuzumab), antibody-drug conjugates (e.g. gemtuzumab), anti-CD33 antibody, a TRAIL-1 ligand, velcade, and pharmaceutically acceptable salts, acids or derivatives of any of the above.

In various embodiments, the disclosure pertains to cancer treatments including, without limitation, those described in US Patent Publication No. US 2012-0225851 and International Patent Publication No. WO 2012/122370.

### Detection Methods

In some embodiments, the cell's apoptotic state is determined by measuring the amount of cytochrome C release from the mitochondria, a marker of apoptosis. This can be measured using standard techniques known in the art (See for example, Current Protocols in Molecular Biology, Greene Publ. Assoc. Inc. & John Wiley & Sons, Inc., Boston, Mass., 1993).

In some embodiments, the cell's apoptotic state is determined by measuring the amount of the cell's mitochondrial outer membrane permeabilization (MOMP). Without being bound by theory, the degree of MOMP indicates the apoptotic state of the cell. This can be performed using standard techniques known in the art, including those described in Bogenberger et al. (Leukemia et al. (2014)). In a non-limiting example, cells are permeabilized and incubated with a fixable mitochondrial dye and BH3 peptides and/or mimetics with dimethyl sulfoxide or carbonyl cyanide m-chlorophenyl hydrazone (CCCP) and the degree of staining is measured.

In some embodiments, the methods disclosed herein use one or more fluorescent dyes that stain mitochondria and/or accumulate based upon mitochondrial membrane potential. In some embodiments, cell permeant dyes that permanently bind to the mitochondria are used. In some embodiments, the cells may be fixed after exposure to the dye to allow later processing and analysis. In some embodiments, the fluorescent dye can be detected after the sample has been fixed and stored.

Without being bound by theory, the use of conventional fluorescent stains for mitochondria, such as rhodamine 123, tetramethylrosamine and JC-1, are not appropriate in the presently disclosed methods because they are washed out of the cells once the mitochondrion's membrane potential is lost and require the signal be read shortly after the application of the dye. Since these conventional dyes cannot be stabilized in the mitochondria by a fixative step, their use in detecting the apoptotic state of solid tumor samples is limited. To overcome this limitation, the method disclosed herein uses alternative mitochondrial membrane potential-dependent dyes that allow the cells and/or tissue to be fixed after the signal is established. These dyes contain a mildly thiol-reactive chloromethyl moiety which appears to be responsible for keeping the dye associated with the mitochondria after fixation. They are concentrated by active mitochondria and retained during cell fixation.

In some embodiments, the measurement of fixed potentiometric dyes after fixation is a readout indicating the mitochondrial membrane integrity response to the BH3 peptides and/or BH3 mimetic reagents. These read outs are combined with other clinical variants or measurements from the natural state of the cancer cell and applied to algorithms that indicate the likelihood of patient response to chemotherapies.

In some embodiments, the cell-permeant MitoTracker® dyes are used to label the mitochondria. MitoTracker® dyes such as MitoTracker® Orange CMTMRos, MitoTracker® Orange CM-H2TMRos, MitoTracker® Red CMXRos, MitoTracker® Red CM-H2XRos, MitoTracker® Deep Red, MitoTracker® Green FM, and MitoTracker® Red FM are used to fluorescently label the mitochondria. To label mitochondria, cells are incubated with the MitoTracker® dyes, which passively diffuse across the plasma membrane and accumulate in active mitochondria. Once the mitochondria are labeled, the cells can be treated with a fixative (e.g. an aldehyde-based fixative) to allow further processing of the sample. MitoTracker® probes are also retained after permeabilization with some detergents during subsequent processing steps (e.g., immunocytochemistry or in situ hybridization). Fluorescence can be measured using any appropriate means in the art. In some embodiments, the fluorescence of the stained and fixed sample is measured using a LI-COR imager. In some embodiments, the fluorescence of the stained and fixed sample is measured using microscopy.

The LI-COR IR imaging system is well suited for quantitative signal analysis in fixed cells and solid tumor thin sections. The system provides several advantages over microscopy or high throughput western blotting. The detection system uses near-infrared (IR) fluorophores (670-1100 nm) that have a distinct advantage over visible dyes, in that very low background fluorescence at longer wavelengths provides an excellent signal-to-noise ratio. Common visible fluorophores cannot be used effectively for direct protein detection on membranes and in plastic plates because of their high background fluorescence in the visible range. In this system antibodies labeled with IR dyes at different wavelengths are used for detection of multiple targets. The imager simultaneously detects two distinct wavelengths.

In various embodiments, the present methods include evaluating a presence, absence, or level of a protein, a protein complex (e.g. dimer), and/or a nucleic acid. In various embodiments, the present methods include evaluating a presence, absence, or level of a protein, a protein complex (e.g. dimer), and/or a nucleic acid which can enhance the specificity and/or sensitivity of mitochondrial profiling. In some embodiments, a marker for patient response is evaluated. In some embodiments, the present methods include measurement using one or more of immunohistochemical staining, Western blotting, in cell Western assay, immunofluorescent staining, ELISA, LI-COR, and/or fluorescent activating cell sorting (FACS), or any other method described herein or known in the art. The present methods may include contacting an antibody with a tumor specimen (e.g. biopsy or tissue or body fluid) to identify an epitope that is specific to the tissue or body fluid and that is indicative of a state of a cancer. In some embodiments, the antibody specifically binds a Bcl-2 heterodimer. In some embodiments, the antibody specifically binds a Bcl-xL/Bim heterodimer. In some embodiments, the antibody is anti-HSBXB. In some embodiments, the antibody is an anti-Bcl-xL antibody.

There are generally two strategies used for detection of epitopes on antigens in body fluids or tissues, direct methods and indirect methods. The direct method includes a one-step staining, and may involve a labeled antibody (e.g. FITC conjugated antiserum) reacting directly with the antigen in a body fluid or tissue sample. The indirect method includes an unlabeled primary antibody that reacts with the body fluid or tissue antigen, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et al. (Antibodies, Cold Spring Harbor Laboratory, NY, 1988), Harlow et al, (Using Antibodies, A Laboratory Manual, Cold Spring Harbor Laboratory, NY, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, Calif.).

In various embodiments, antibodies include whole antibodies and/or any antigen binding fragment (e.g., an antigen-binding portion) and/or single chains of these (e.g. an antibody comprising at least two heavy (H) chains and two light (L) chains inter-connected by disulfide bonds, an Fab fragment, a monovalent fragment consisting of the V_{L}, V_{H}, C_{L} and CH1 domains; a F(ab)₂ fragment, a bivalent fragment including two Fab fragments linked by a disulfide bridge at the hinge region; a Fd fragment consisting of the V_{H} and CH1 domains; a Fv fragment consisting of the V_{L} and V_{H} domains of a single arm of an antibody; and the like). In various embodiments, polyclonal and monoclonal antibodies are useful, as are isolated human or humanized antibodies, or functional fragments thereof.

Standard assays to evaluate the binding ability of the antibodies toward the target of various species are known in the art, including for example, ELISAs, western blots and RIAs. The binding kinetics (e.g., binding affinity) of antibodies also can be assessed by standard assays known in the art, such as by Biacore analysis.

In other embodiments, the measurement includes evaluating a presence, absence, or level of a nucleic acid. A person skilled in the art will appreciate that a number of methods can be used to detect or quantify the DNA/RNA levels of appropriate markers.

Gene expression can be measured using, for example, low-to-mid-plex techniques, including but not limited to reporter gene assays, Northern blot, fluorescent in situ hybridization (FISH), and reverse transcription PCR (RT-PCR). Gene expression can also be measured using, for example, higher-plex techniques, including but not limited, serial analysis of gene expression (SAGE), DNA microarrays. Tiling array, RNA-Seq/whole transcriptome shotgun sequencing (WTSS), high-throughput sequencing, multiplex PCR, multiplex ligation-dependent probe amplification (MLPA), DNA sequencing by ligation, and Luminex/XMAP. A person skilled in the art will appreciate that a number of methods can be used to detect or quantify the level of RNA products of the biomarkers within a sample, including arrays, such as microarrays, RT-PCR (including quantitative PCR), nuclease protection assays and Northern blot analyses.

### Cancers and Patients

A cancer or tumor refers to an uncontrolled growth of cells and/or abnormal increased cell survival and/or inhibition of apoptosis which interferes with the normal functioning of the bodily organs and systems. A subject that has a cancer or a tumor is a subject having objectively measurable cancer cells present in the subject's body. Included in this disclosure are benign and malignant cancers, as well as dormant tumors or micrometastatses. Cancers which migrate from their original location and seed vital organs can eventually lead to the death of the subject through the functional deterioration of the affected organs.

In various embodiments, the disclosure is applicable to pre-metastatic cancer, or metastatic cancer. Metastasis refers to the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. Metastases are often detected through the sole or combined use of magnetic resonance imaging (MRI) scans, computed tomography (CT) scans, blood and platelet counts, liver function studies, chest X-rays and bone scans in addition to the monitoring of specific symptoms.

The methods described herein are directed toward the prognosis of cancer, diagnosis of cancer, treatment of cancer, and/or the diagnosis, prognosis, treatment, prevention or amelioration of growth, progression, and/or metastases of malignancies and proliferative disorders associated with increased cell survival, or the inhibition of apoptosis. In some embodiments, the cancer is a solid tumor, including, but not limited to, non-small lung cell carcinoma, ovarian cancer, breast cancer, prostate cancer, lung cancer, pancreatic cancer, hepatic cancer, brain cancer, and/or melanoma.

In some embodiments, the disclosure relates to one or more of the following cancers: adrenocortical carcinoma, AIDS-related cancers, anal cancer, appendix cancer, astrocytoma (e.g. childhood cerebellar or cerebral), basal-cell carcinoma, bile duct cancer, bladder cancer, bone tumor (e.g. osteosarcoma, malignant fibrous histiocytoma), brainstem glioma, brain cancer, brain tumors (e.g. cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumors, central cerebellar astrocytoma, cervical cancer, chronic myeloproliferative disorders, colon cancer, desmoplastic small round cell tumor, endometrial cancer, ependymoma, esophageal cancer, Ewing's sarcoma, extracranial germ cell tumor, extragonadal germ cell tumor, extrahepatic bile duct cancer, eye cancer, gallbladder cancer, gastric (stomach) cancer, gastrointestinal stromal tumor (GIST), germ cell tumor (e.g. extracranial, extragonadal, ovarian), gestational trophoblastic tumor, gliomas (e.g. brain stem, cerebral astrocytoma, visual pathway and hypothalamic), gastric carcinoid, head and neck cancer, heart cancer, hepatocellular (liver) cancer, hypopharyngeal cancer, hypothalamic and visual pathway glioma, intraocular melanoma, islet cell carcinoma (endocrine pancreas), kidney cancer (renal cell cancer), laryngeal cancer, lip and oral cavity cancer, liposarcoma, liver cancer, lung cancer (e.g. non-small cell, small cell), medulloblastoma, melanoma, Merkel cell carcinoma, mesothelioma, metastatic squamous neck cancer, mouth cancer, multiple endocrine neoplasia syndrome, mycosis fungoides, myelodysplastic syndromes, myelodysplastic/myeloproliferative diseases, chronic, nasal cavity and paranasal sinus cancer, nasopharyngeal carcinoma, neuroblastoma, non-small cell lung cancer, oral cancer, oropharyngeal cancer, osteosarcoma, ovarian cancer, pancreatic cancer, pancreatic cancer, paranasal sinus and nasal cavity cancer, parathyroid cancer, penile cancer, pharyngeal cancer, pheochromocytoma, pineal astrocytoma and/or germinoma, pineoblastoma and supratentorial primitive neuroectodermal tumors, pituitary adenoma, pleuropulmonary blastoma, prostate cancer, rectal cancer, renal cell carcinoma (kidney cancer), renal pelvis and ureter, retinoblastoma, rhabdomyosarcoma, salivary gland cancer, sarcoma (e.g. Ewing family, Kaposi, soft tissue, uterine), Sézary syndrome, skin cancer (e.g. nonmelanoma, melanoma, merkel cell), small cell lung cancer, small intestine cancer, soft tissue sarcoma, squamous cell carcinoma, squamous neck cancer, stomach cancer, supratentorial primitive neuroectodermal tumor, testicular cancer, throat cancer, thymoma and thymic carcinoma, thyroid cancer, trophoblastic tumors, ureter and renal pelvis cancers, urethral cancer, uterine cancer, uterine sarcoma, vaginal cancer, visual pathway and hypothalamic glioma, vulvar cancer, Waldenstrom macroglobulinemia, and Wilms tumor. In some embodiments, the cancer is breast cancer, non-small lung cancer, carcinoma, ovarian cancer, prostate cancer, lung cancer, pancreatic cancer, hepatic cancer, brain cancer and/or melanoma.

The term subject, as used herein unless otherwise defined, is a mammal, e.g., a human, mouse, rat, hamster, guinea, pig, dog, cat, horse, cow, goat, sheep, pig, or non-human primate, such as a monkey, chimpanzee, or baboon. The terms “subject” and “patient” are used interchangeably.

### Specimens

The tumor cells, samples, and/or specimens are obtained from a solid tumor. In some embodiments, information is collected from biopsied patient tumor cells, samples, and/or specimens that are treated with mitochondrial membrane disrupting reagents, stained with fixable mitochondrial membrane potential dye and fixed. In some embodiments, the sample is a tissue thin-section. In some embodiments, the sample is not a cell suspension. Analysis of the data collected from this method is applied to an algorithm including measurements of other naturally occurring states of the cancer cell. The taken together the algorithm readout is used as a predictive biomarker for patient response to treatment.

In some embodiments, the present disclosure includes the measurement of a tumor cell, sample, and/or specimen, including biopsy or surgical specimen samples. In some embodiments, the cell, sample, and/or specimen is selected from a frozen tumor tissue specimen, cultured cells, circulating tumor cells, and/or a formalin-fixed paraffin-embedded tumor tissue specimen. In some embodiments, the biopsy is a human biopsy. In some cases these sections will be formalin-fixed paraffin-embedded post biopsy.

In some embodiments, the tumor cell, sample, and/or antigen is a cryosection. As is known in the art, a cryosection may employ a cryostat. The surgical sample and/or specimen is placed on a metal tissue disc which is then secured in a chuck and frozen rapidly to about −20° C. to about −30° C. The sample and/or specimen is embedded in a gel like medium consisting of, for example, poly ethylene glycol and polyvinyl alcohol. The frozen tissue is cut frozen with the microtome portion of the cryostat, and the section is optionally picked up on a glass slide and stained. In some embodiments, the cryosection is between 1-50 μM thick. In some embodiments, the cryosection is between about 1-5 μM thick, about 1-10 μM thick, about 1-15 μM thick, about 1-20 μM thick, about 1-30 μM thick, and about 1-40 μM thick. In some embodiments, the cryosection is assayed for mitochondrial profiling, the mitochondria are stained with a fixable dye, and the cryosection is fixed.

In some embodiments, the biopsy is a formalin-fixed paraffin-embedded (FFPE) tumor tissue specimen. As is known in the art, a biopsy specimen may be placed in a container with formalin (a mixture of water and formaldehyde) or some other fluid to preserve it. The tissue specimen and/or sample may be placed into a mold with hot paraffin wax. The wax cools to form a solid block that protects the tissue. This paraffin wax block with the embedded tissue is placed on a microtome, which cuts very thin slices of the tissue. In some embodiments, the tumor cells, sample, and/or specimen is fixed with an aldehyde-based fixative.

In certain embodiments, the tumor sample, and/or specimen (or biopsy) contains less than 100 mg of tissue, or in certain embodiments, contains about 50 mg of tissue or less. The tumor sample and/or specimen (or biopsy) may contain from about 20 mg to about 50 mg of tissue, such as about 35 mg of tissue.

The tissue may be obtained, for example, as one or more (e.g., 1, 3, 4, or 5) needle biopsies (e.g., using a 14-gauge needle or other suitable size). In some embodiments, the biopsy is a fine-needle aspiration in which a long, thin needle is inserted into a suspicious area and a syringe is used to draw out fluid and cells for analysis. In some embodiments, the biopsy is a core needle biopsy in which a large needle with a cutting tip is used during core needle biopsy to draw a column of tissue out of a suspicious area. In some embodiments, the biopsy is a vacuum-assisted biopsy in which a suction device increases the amount of fluid and cells that is extracted through the needle. In some embodiments, the biopsy is an image-guided biopsy in which a needle biopsy is combined with an imaging procedure, such as, for example, X ray, computerized tomography (CT), magnetic resonance imaging (MRI) or ultrasound. In other embodiments, the sample may be obtained via a device such as the MAMMOTOME® biopsy system, which is a laser guided, vacuum-assisted biopsy system for breast biopsy.

In certain embodiments the cells, sample, and/or specimen is a human tumor-derived cell line. In certain embodiments, the cells, sample, and/or specimen is a cancer stem cell. In other embodiments, the cells, sample, and/or specimen is derived from the biopsy of a solid tumor, such as, for example, a biopsy of a colorectal, breast, prostate, lung, pancreatic, renal, or ovarian primary tumor.

In certain embodiments, the cells, sample, and/or specimen is of epithelial origin. In some embodiments, the epithelial specimen is enriched by selection from a biopsy sample with an anti-epithelial cell adhesion molecule (EpCAM) or other epithelial cell binding antibody bound to solid matrix or bead.

In certain embodiments, the cells, sample, and/or specimen is of mesenchymal origin. In some embodiments, the mesenchymal specimen is enriched by selection from a biopsy sample with a neural cell adhesion molecule (N-CAM) or neuropilin or other mesenchymal cell binding antibody bound to a solid matrix or bead.

### Clinical Factors and Additional Biomarkers

In some embodiments, a clinical factor that provides patient response information in combination with a solid tumor mitochondrial profiling study may not be linked to apoptosis. In some embodiments, a clinical factor that provides patient response information in combination with a solid tumor mitochondrial profiling study may be linked to apoptosis. In some embodiments, a clinical factor is non-apoptosis affecting. In some embodiments, a clinical factor is apoptosis affecting.

In some embodiments, the clinical factor is age. In some embodiments, the patient age profile is classified as over about 10, or over about 20, or over about 30, or over about 40, or over about 50, or over about 60, or over about 70, or over about 80 years old.

In some embodiments, the clinical factor is cytogenetic status. In some cancers, such as Wilms tumor and retinoblastoma, for example, gene deletions or inactivations are responsible for initiating cancer progression, as chromosomal regions associated with tumor suppressors are commonly deleted or mutated. For example, deletions, inversions, and translocations are commonly detected in chromosome region 9p21 in gliomas, non-small-cell lung cancers, leukemias, and melanomas. Without wishing to be bound by theory, these chromosomal changes may inactivate the tumor suppressor cyclin-dependent kinase inhibitor 2A. Along with these deletions of specific genes, large portions of chromosomes can also be lost. For instance, chromosomes 1p and 16q are commonly lost in solid tumor cells. Gene duplications and increases in gene copy numbers can also contribute to cancer and can be detected with transcriptional analysis or copy number variation arrays. For example, the chromosomal region 12q13-q14 is amplified in many sarcomas. This chromosomal region encodes a binding protein called MDM2, which is known to bind to a tumor suppressor called p53. When MDM2 is amplified, it prevents p53 from regulating cell growth, which can result in tumor formation. Further, certain breast cancers are associated with overexpression and increases in copy number of the ERBB2 gene, which codes for human epidermal growth factor receptor 2. Also, gains in chromosomal number, such as chromosomes 1 q and 3q, are also associated with increased cancer risk. Further, mutations in the genes MLL, AML/ETO, Flt3-ITD, NPM1 (NPMc+), CEBPα, IDH1, IDH2, RUNX1, ras, and WT1 in the epigenetic modifying genes TET2 and ASXL, as well as changes in the cell signaling protein profile are associated with increased cancer risk.

Cytogenetic status can be measured in a variety of manners known in the art. For example, FISH, traditional karyotyping, and virtual karyotyping (e.g. comparative genomic hybridization arrays, CGH and single nucleotide polymorphism arrays) may be used. For example, FISH may be used to assess chromosome rearrangement at specific loci and these phenomena are associated with disease risk status. In some embodiments, the cytogenetic status is favorable, intermediate, or unfavorable.

In some embodiments, the clinical factor is performance. Performance status can be quantified using any system and methods for scoring a patient's performance status are known in the art. The measure is often used to determine whether a patient can receive chemotherapy, adjustment of dose adjustment, and to determine intensity of palliative care. There are various scoring systems, including the Karnofsky score and the Zubrod score. Parallel scoring systems include the Global Assessment of Functioning (GAF) score, which has been incorporated as the fifth axis of the Diagnostic and Statistical Manual (DSM) of psychiatry. Higher performance status (e.g., at least 80%, or at least 70% using the Karnofsky scoring system) may indicate treatment to prevent progression of the disease state, and enhance the patient's ability to accept chemotherapy and/or radiation treatment. For example, in these embodiments, the patient is ambulatory and capable of self care. In other embodiments, the evaluation is indicative of a patient with a low performance status (e.g., less than 50%, less than 30%, or less than 20% using the Karnofsky scoring system), so as to allow conventional radiotherapy and/or chemotherapy to be tolerated. In these embodiments, the patient is largely confined to bed or chair and is disabled even for self-care.

The Karnofsky score runs from 100 to 0, where 100 is “perfect” health and 0 is death. The score may be employed at intervals of 10, where: 100% is normal, no complaints, no signs of disease; 90% is capable of normal activity, few symptoms or signs of disease, 80% is normal activity with some difficulty, some symptoms or signs; 70% is caring for self, not capable of normal activity or work; 60% is requiring some help, can take care of most personal requirements; 50% requires help often, requires frequent medical care; 40% is disabled, requires special care and help; 30% is severely disabled, hospital admission indicated but no risk of death; 20% is very ill, urgently requiring admission, requires supportive measures or treatment; and 10% is moribund, rapidly progressive fatal disease processes.

The Zubrod scoring system for performance status includes: 0, fully active, able to carry on all pre-disease performance without restriction; 1, restricted in physically strenuous activity but ambulatory and able to carry out work of a light or sedentary nature, e.g., light house work, office work; 2, ambulatory and capable of all self-care but unable to carry out any work activities, up and about more than 50% of waking hours; 3, capable of only limited self-care, confined to bed or chair more than 50% of waking hours; 4, completely disabled, cannot carry on any self-care, totally confined to bed or chair; 5, dead.

In some embodiments, the clinical factor is histological subclass. In some embodiments, histological samples of tumors are graded according to Elston & Ellis, Histopathology, 1991, 19:403-10, the contents of which are hereby incorporated by reference in their entirety.

In some embodiments, the clinical factor is gender. In some embodiments, the gender is male. In other embodiments, the gender is female.

In some embodiments, the clinical factor is disease stage. By way of non-limiting example, using the overall stage grouping, Stage I cancers are localized to one part of the body; Stage II cancers are locally advanced, as are Stage III cancers. Whether a cancer is designated as Stage II or Stage III can depend on the specific type of cancer. In one non-limiting example, Hodgkin's disease, Stage II indicates affected lymph nodes on only one side of the diaphragm, whereas Stage III indicates affected lymph nodes above and below the diaphragm. The specific criteria for Stages II and III therefore differ according to diagnosis. Stage IV cancers have often metastasized, or spread to other organs or throughout the body.

In other embodiments, the method further includes a measurement of an additional biomarker selected from mutational status, single nucleotide polymorphisms, steady state protein levels, and dynamic protein levels which can add further sensitivity and/or specificity to the analysis. In other embodiments, the method further includes predicting a clinical response in the patient. In other embodiments, the clinical response is about 1, about 2, about 3, or about 5 year progression/event-free survival.

In some embodiments, a subject is likely to be afflicted by cancer if the subject is characterized by a high risk for a cancer. In some embodiments, a subject is likely to be afflicted by cancer if the subject is characterized by a genetic predisposition to a cancer. In some embodiments, a genetic predisposition to a cancer is a genetic clinical factor, as is known in the art. Such clinical factors may include, by way of example, HNPCC, MLH1, MSH2MSH6, PMS1, PMS2 for at least colon, uterine, small bowel, stomach, urinary tract cancers. In some embodiments, a subject is likely to be afflicted by cancer if the subject is characterized by a previous episode of a cancer (e.g. new cancers and/or recurrence). In some embodiments, the subject has been afflicted with 1, or 2, or 3, or 4, or 5, or 6, previous episodes of cancer. In some embodiments, a subject is likely to be afflicted by cancer if the subject is characterized by a family history of a cancer. In some embodiments, a parent and/or grandparent and/or sibling and/or aunt/uncle and/or great aunt/great uncle, and/or cousin has been or is afflicted with a cancer. In some embodiments, a subject is likely to be afflicted by cancer if the subject is characterized by exposure to a cancer-inducing agent (e.g. an environmental agent). For example, exposing skin to strong sunlight is a clinical factor for skin cancer. By way of example, smoking is a clinical factor for cancers of the lung, mouth, larynx, bladder, kidney, and several other organs. In some embodiments, the subject is likely to be afflicted by cancer based on pharmacogenomic information (the effect of genotype on the pharmacokinetic, pharmacodynamic or efficacy profile of a therapeutic).

Further, in some embodiments, any one of the following clinical factors may be useful in the methods described herein: race and ethnicity; features of the certain tissues; various benign conditions (e.g. non-proliferative lesions); previous chest radiation; carcinogen exposure and the like.

Further still, in some embodiments, any one of the following clinical factors may be useful in the methods described herein: one or more of a cell surface marker CD33, a cell surface marker EpCAM, CEA, IGFR1, EGRF, CD34, or HER2 expression level, a p53 mutation status, a K-Ras mutational status, a phosphorylation state of MEK-1 kinase, and phosphorylation of serine at position 70 of Bcl-2.

In some embodiments, the clinical factor is expression levels of the cytokines, including, without limitation, interleukin-6. In some embodiments, interleukin-6 levels will correlate with likelihood of response in cancer patients, including a poor patient prognosis or a good patient prognosis.

### Kits

The disclosure also provides kits that can simplify the evaluation of tumor or cancer cell specimens. A typical kit of the disclosure contains various reagents including, for example, one or more agents to detect cancer cell response to one or more BH3 peptides or BH3 mimetic compounds. A kit may also include one or more of reagents for detection, including those useful in various detection methods, such as, for example, antibodies and/or fixable mitochondrial dyes. The kit can further contain materials necessary for the evaluation, including welled plates, syringes, and the like. The kit can further include a label or printed instructions instructing the use of described reagents. The kit can further include a treatment to be tested.

It should be understood that singular forms such as “a,” “an,” and “the” are used throughout this application for convenience, however, except where context or an explicit statement indicates otherwise, the singular forms are intended to include the plural. Further, it should be understood that every journal article, patent, patent application, publication, and the like that is mentioned herein is hereby incorporated by reference in its entirety and for all purposes. All numerical ranges should be understood to include each and every numerical point within the numerical range, and should be interpreted as reciting each and every numerical point individually. The endpoints of all ranges directed to the same component or property are inclusive, and intended to be independently combinable.

The term “about” when used in connection with a referenced numeric indication means the referenced numeric indication plus or minus up to 10% of that referenced numeric indication. For example, the language “about 50” covers the range of 45 to 55.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

This disclosure is further illustrated by the following non-limiting examples.

### INCORPORATION BY REFERENCE

This application incorporates by reference for all purposes the entirety of the following: U.S. Pat. No. 8,168,755 filed May 7, 2009, which claims benefit of U.S. Application No. 61/051,206 filed May 7, 2008; Serial No. PCT/US2013/040585 filed on May 10, 2013, which claims benefit of U.S. Application No. 61/780,252 filed Mar. 13, 2013 and U.S. Application No. 61/645,253 filed May 10, 2012; Serial No. PCT/US2014/049420 filed Aug. 1, 2014, which claims benefit of U.S. Application No. 61/861,009 filed Aug. 1, 2014; Serial No. PCT/US2014/047307 filed Jul. 18, 2014, which claims benefit of U.S. Application No. 61/847,750 filed Jul. 18, 2013; and Serial No. PCT/US2014/063121, filed Oct. 30, 2014, and U.S. Application No. 61/897,547 filed Oct. 30, 2014.

All publications, patents, and patent publications cited are incorporated by reference herein in their entirety for all purposes.

### EXAMPLES

### Example 1

### Mitochondrial Profiling in Samples Taken from Solid Tumor Oncology Patient-Based Cohorts

Briefly, in this assay, the tumor specimen is prepared, subjected to various BH3 peptides, or BH3 mimetic compounds and the mitochondria stained with a fixable dye that fluoresces upon oxidation (indicating an increase in MOMP). Detection of the fluorescent signal (through fluorescent microscopy or LICOR imaging) detects whether MOMP has occurred. The percent of mitochondrial priming is determined via the equation disclosed herein. Multivariate analysis, taking into account the percent mitochondrial priming with and without other clinical or pathological factors, is used to predict the efficacy of one or more cancer treatments in the patient.

In addition to testing the tumor specimen, the following controls are used: 1) DMSO only—in place of a BH3 peptide and/or mimetics, and 2) carbonyl cyanide m-chlorophenylhydrazone (CCCP) for an apoptosis positive control (these cells will be MitoTracker® Orange negative since the dye only fluoresces upon oxidation).

Tissues from core needle biopsies from solid tumors are frozen in DMSO-containing freezing media (or equivalent) in cryogenic storage vials which are frozen in a controlled rate freezing apparatus. Tissues are stored in liquid nitrogen until further processing.

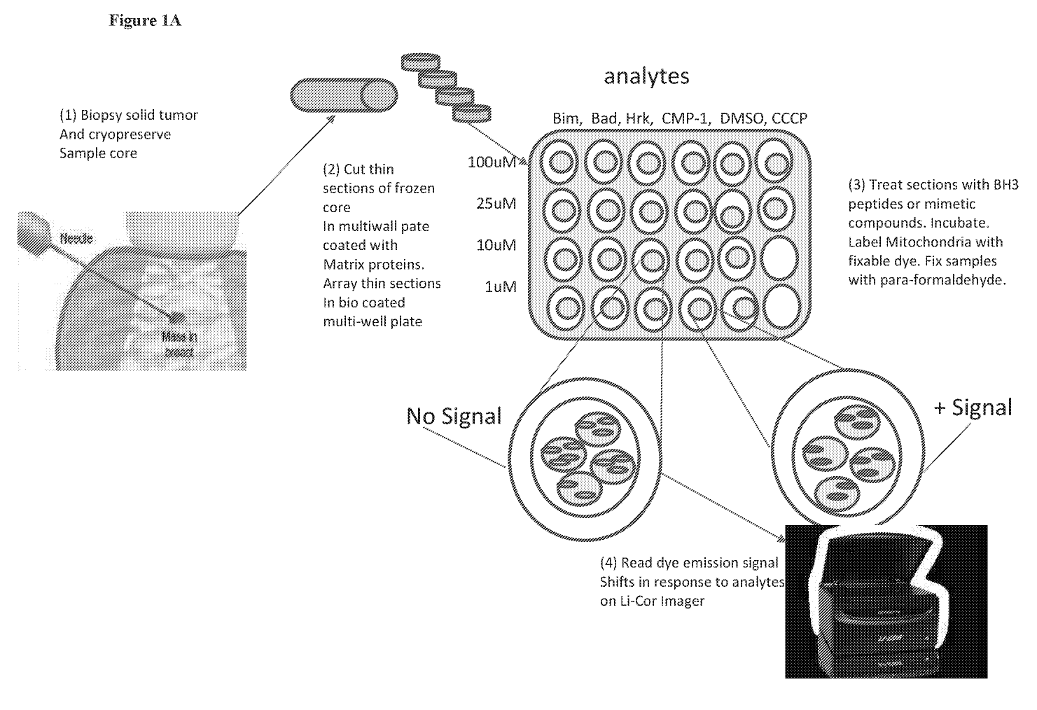

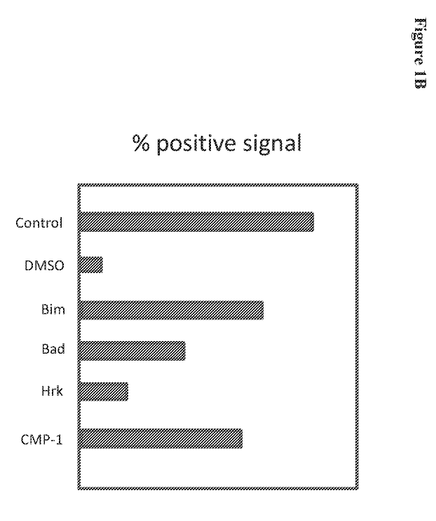

FIG. 1A shows a representative mitochondrial profiling assay scheme. For processing, the entire frozen sample is removed from the vial and cut using a cryostat into 5-15 μm sections. These cut sections are then transferred into individual wells of a multi-well plate, and each tissue section is washed with 200 μL of PBS (or equivalent). 100 μL of a dye that permanently labels dead cells (e.g. Fixable Viability Dye eFluor® 660) is added to each well, and incubated for 20 minutes at room temperature to exclude dead cells. The tissue is washed three times with 200 μL of PBS.

After the sample is prepared, mitochondrial profiling is performed by adding the BH3 peptides or peptide mimetic compounds and digitonin/oligomycin to the individual wells containing tissue sections. The plate is incubated for two hours at room temperature in the dark, and the tissue is then washed three times with 200 μL of PBS.

After exposure to the BH3 panel, the cells are stained with MitoTracker® Orange CMTMRos at a concentration of 25-500 nM for 15-45 minutes in PBS. This dye will fluoresce when it is oxidized; in its reduced state, it is colorless. Since MOMP increases the oxidation of mitochondria, the detection of an orange fluorescent signal indicates MOMP has occurred. Other potentiometric dyes that can withstand fixation would be used as well.

The tissue is then washed three times with 200 μL of PBS, and 100 μL of DAPI at a concentration of 300 nM in PBS is added. The plate is incubated for 10 minutes at room temperature. The tissue is then washed three times with 200 μL of PBS.

The tissue is fixed with 100 μL of 2% PFA for 20 minutes at room temperature and then washed three times with PBS. Fluorescent images are captured directly in the wells using a fluorescent microscopy system using either a lamp or lasers, which is capable of high content screening (HCS) and high content analysis (HCA). Multiple fluorescence filters will be used: the DAPI filter cube detects DAPI binding to nucleic acids and indicates a permeabilized cell, the MitoTracker Orange filter cube detects the fluorescence signal from the MitoTracker Orange dye which indicates mitochondria undergoing MOMP, and the APC filter cube is used as a negative selection to identify viable cells. The. APC filter cube is used to dye dead cells (e.g. by using BioLegend: Zombie Red™ fixable viability dye or similar) and a lack of signal (negative cells) indicates viable cells. The viable cells are then contoured based on the DAPI fluorescence and the MitoTracker Orange fluorescence signal is integrated in the region surrounding the nucleus. To determine the degree of MOMP in the sample, the fluorescence intensity of the MitoTracker Orange channel from the individual events (cells) is measured and is used to calculate a median fluorescence intensity for the tumor cells. Then using the following priming equation, the percentage priming of each peptide can be determined:

$$\% Priming = [ 100 * ( \frac{DMSO AUC - {Peptide}_{1} AUC}{DMSO AUC - {CCCP}_{avg} AUC} ) ] {Peptide}_{1} + [ 100 * ( \frac{DMSO AUC - {Peptide}_{2} AUC}{DMSO AUC - {CCCP}_{avg} AUC} ) ] {Peptide}_{2} + … / ( n peptides )$$

Alternatively, the LI-COR imaging system may be used. The percent of MitoTracker Orange positive signal (e.g. fluorescence) can be detected by LI-COR giving a measure of MOMP in the cancer cells. The intensity of DAPI fluorescence will represent the total cellular fluorescence contribution and will be adjusted based on the viability dye fluorescence (APC channel). The positive control for dead cells will be used to normalize the fraction alive in this scenario. Then the orange fluorescence will be adjusted based on the alive cellular total calculated from the DAPI and viability dye fluorescence. FIG. 1B shows a representative analyte response profile comparing the percent positive signal among the different peptides tested in the multi-well plate.

The signal intensity obtained from the various peptides and controls in the mitochondrial profiling assay can be used in the algorithm to determine the mitochondrial priming state of the cancer cells. The mitochondrial priming state can be used to inform the clinician of the Bcl-2 heterodimers in play in the cancer tested, and when taken into consideration with other clinical factors (e.g. using multivariate analysis), can predict a patient's response to a cancer treatment.

### Example 2

### Detection of Bcl-2 Heterodimers in Solid Minor Cells Using an Antibody

In addition to detecting MOMP in a sample to elucidate which Bcl-2 heterodimers play a role in the apoptotic state of the cancer, these heterodimers may be directly detected in a cancer sample. Here, an antibody that binds to a particular Bcl-2 heterodimer is applied to the sample, and then detected through immunofluorescence.

Antibodies have been developed that specifically bind to Bcl-2 heterodimers (see, for example, U.S. Pat. No. 8,168,755). These antibodies detect different Bcl-2 heterodimers present in a particular cancer, thereby informing the clinician of which BH3 peptides are influencing apoptosis in a particular cancer cell. This information can be used to help direct treatment and clinical decisions. The results from the mitochondrial profiling described in Example 1 can be compared with the results of antibody based heterodimer detection in samples taken from the same tumor and/or patient to increase the sensitivity and specificity of the analysis.

Immunofluorescence staining is used to detect Bcl-2 heterodimers in solid tumors. As described in Example 1, tissues from core needle biopsies from solid tumors are frozen in DMSO-containing freezing media (or equivalent) in cryogenic storage vials which are frozen in a controlled rate freezing apparatus. Tissues are stored in liquid nitrogen until further processing. The entire frozen sample is removed from the vial and cut using a cryostat into 5-15 μm sections. These cut sections are then transferred into individual wells of a multi-well plate, and each tissue section is washed with 200 μL of PBS (or equivalent). 100 μL of a dye that permanently labels dead cells (e.g. Fixable Viability Dye eFluor® 660) is added to each well, and incubated for 20 minutes at room temperature to exclude dead cells. For a positive control for the viability dye, a tissue section is heated to 65° C. for one minute, and then placed on ice for one minute. The tissue is washed three times with 200 μL of PBS. Alternatively, depending on the stability of the antigens in question, tissue sections or core needle biopsies may be fixed in 2% PFA, dehydrated with ethanol, and then embedded in paraffin. Sections (5-15 μm) will be prepared using a microtome and transferred into individual wells of 96-well plates. Paraffin is removed with xylenes, and the tissue sections are rehydrated. Antigen retrieval is performed using heat-induced epitope retrieval proteolytic-induced epitope retrieval, or an alternate method.

The tissue is then fixed with 100 μL of 2% PFA for 20 minutes at room temperature. The cells are permeabilized with 0.2% Triton-X 100 diluted in PBS for 10 minutes at room temperature and then washed three times with 100 μL of PBS. The cells are then blocked with 2% Goat Serum diluted in PBS for 15 minutes at room temperature. The block solution is removed and the primary monoclonal antibody that binds to a Bcl-2 heterodimer normal mouse IgG) is applied at a concentration of 1:250 diluted in staining buffer. This is incubated at room temperature for two hours in the dark and then the cells are washed three times with 100 μL of PBS, Goat Anti-mouse AlexaFluor 488 secondary antibody diluted at a concentration of 1:5000 in staining buffer is added, and the cells are washed three times with PBS. The cells are incubated with 100 μL of DAPI at a concentration of 300 nM in PBS for 10 minutes at room temperature.

The cells are imaged with fluorescent microscopy with fluorescent filters including a DAPI filter cube (to detect the cells), AF488 (to detect the bound antibody), and APC (to detect viable cells). APC negative cells, which are viable, are selected for analysis. To detect the bound antibody, the median fluorescence intensity (MFI) of AF488 channel from individual cells in the IgG and heterodimer specific antibody stained tissue sections is measured. Alternatively, the LI-COR IR imaging system is well suited for quantitative signal analysis in fixed cells and solid tumor thin sections.

The detection of particular Bcl-2 heterodimers in a patient's cancer specimen may be used, along with the results of the mitochondrial profiling, and/or other clinical factors to predict treatment efficacy, and to guide clinical decisions for the patient.

### Example 3

### Detection of Bcl-2 Heterodimers in Breast Cancer Cells Using an Antibody

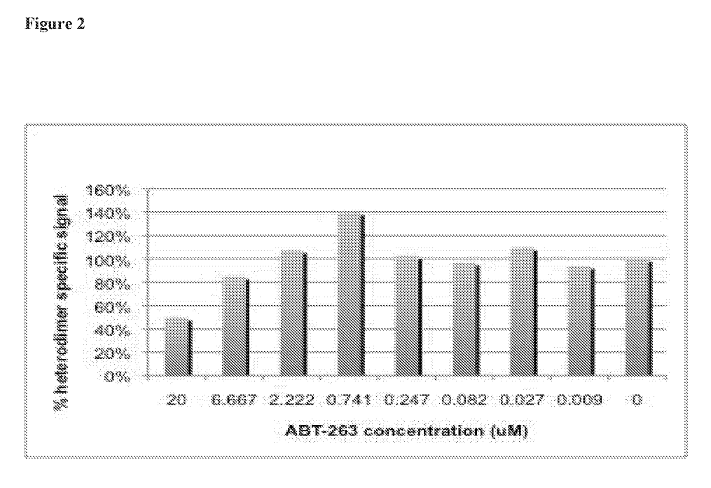

FIG. 2 shows the percent heterodimer specific signal of breast cancer cells using a LI-COR imager. ABT-263 is a pro-apoptotic compound that can be used on whole cells to disrupt BH3 mediated interactions. Disruption of these heterodimers in treated cells is detected as loss of ANTI-HSBXB signal. Here the cells were treated with various concentrations of ABT-263 for 10 hours at 37° C., then rinsed, fixed with 4% PFA, and incubated with ANTI-HSBXB (a Bcl-xL/Bim heterodimer-specific antibody). IRDye 800CW goat anti-mouse antibody was used to detect the heterodimer specific mouse monoclonal antibody, and IRDye 800CW Goat anti-rabbit antibody was used to detect a commercial Bcl-xL rabbit monoclonal antibody. Cell numbers were normalized by using CellTag 700 Stain. We have determined a time window of 10-12 hours where the disruption by ABT-263 can be detected prior to caspase mediated membrane disruption of SKBR3 breast cancer cells. Generally higher concentrations of ABT-263 disrupt the Bcl-2 heterodimers in the cell, thereby decreasing the quantity of ANTI-HSBXB detection (FIG. 2).

### Example 4

### Demonstration of New Methods for Detection of Pre-Apoptatic-Anti-Apopotic Bcl-2 Family Heterodimers in Cancer Cells Using Flow Cytometry and ELISA

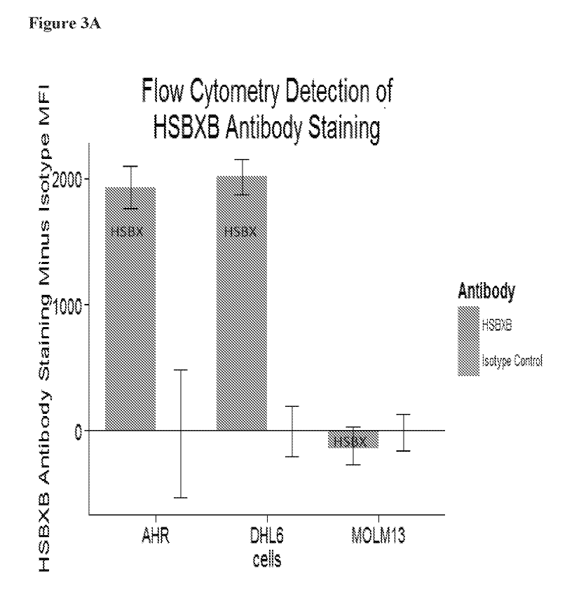

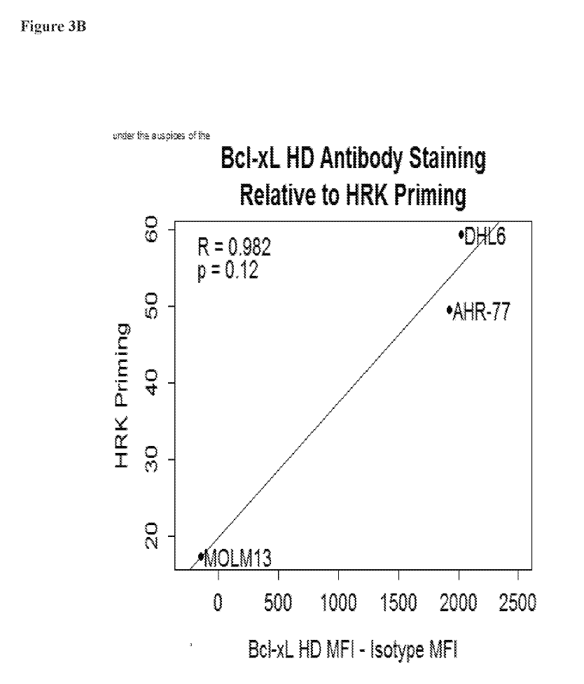

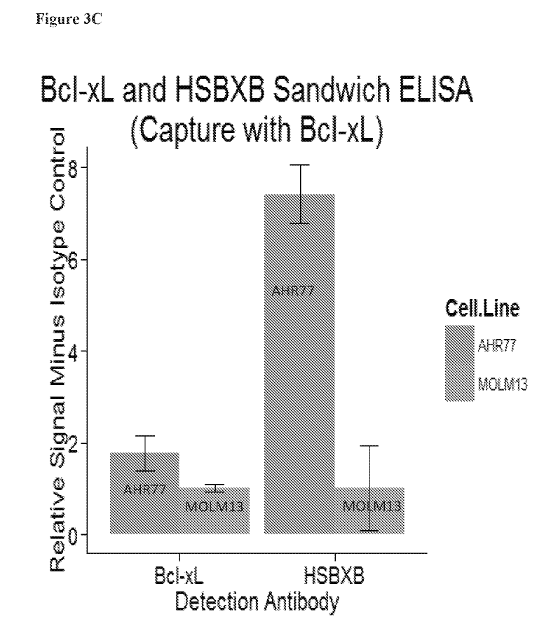

The Bcl-xL/Bim heterodimer (HSBXB) can be detected by flow cytometry using a method for intracellular staining with the HSBXB antibody. Three leukemia cell lines, AHR, DHL6 and MOLM 13 were chosen based on their having differing amounts of Bcl-xL, Bim priming as determined by mitochondrial profiling. Consequently, the response to the Bcl-xL restricted, the Bcl-xL/Bim dimer “priming”, is known to be present at different amounts. A high signal mediated by Hrk-BH3 peptide indicates the existence of Bcl-xL/Bim heterodimer. As seen in FIG. 3, Hrk-BH3 domain peptide indicated degree of Bcl-xL priming. The Molm 13 cells are primed at less than 5% relative to positive control, DHL-6 cells are primed slightly higher and the AHR cells are primed above 60% of positive control in that assay.

The HSBXB signal was assessed by flow cytometry and compared to the Hrk-BH3 peptide signal in the BH3 profiling assay. Here, AHR, DHL6 and MOLM13 cells were incubated on ice for 3 hours, and then washed and incubated with HSBXB antibody or Bcl-xL antibody at 10 ug/ml for 20 minutes, and then washed and stained with secondary Alexa488-conjugated goat anti-mouse. Signals were corrected to IgG-2A isotype or secondary alone control. The Hrk-BH3 signal in the mitochondrial profiling of the three cell lines was plotted against normalized HSBXB FACS signal.

To confirm the correlation to the BH3 priming signal an ELISA assay was performed. Here the anti-Bcl-xL antibody was used to capture of Bcl-xL-Bim complex from RIPA lysed cells. The captured complex was then probed with the HSBXB antibody or the anti-Bcl-xL antibody. As seen in FIG. 3 the correlation held. These data therefore establish two new formats to assess the primed state in solid tumors.

### Example 5

### Secondary Clinical Endpoints: Overall Survival and Event-Free Survival

Solid tumor mitochondrial profiling biomarkers are analyzed for correlation to the secondary clinical endpoints overall survival (OS) and event-free survival (EFS). Further, multivariate analysis with adjustment variables patient age profile and cytogenetic risk status are used to establish correlations for example between solid tumor mitochondrial profiling biomarkers and OS and EFS clinical endpoints.

### Example 6

### Secondary Cancer Cell and Non-Cancer Cell Measurements: Overall Survival and Event Free Survival

Solid tumor mitochondria profiling biomarkers are analyzed for correlation to the measurements of other molecular markers that are indicative of the onset of cancer cell survival of cancer cell death. Such measurements include for example, occurrence of heterodimers consisting of anti-apoptotic and pro-apoptotic Bcl-2 family proteins, phosphorylation of ser-70 on h-Bcl-2, low expression levels of the BH3 only protein Noxa, mutational status of k-ras. Further, multivariate analysis with adjustment variables identified in measurements of genetic mutations in the cancer cells, protein levels, protein modification, and metabolite measurements are used in combination with mitochondria profiling readouts to establish algorithms that correlate with patient response to treatment, as well as OS and EFS clinical endpoints.

What is claimed is:

1. A method for determining a cancer treatment for a patient with a solid tumor, comprising:
a) obtaining cancer cells or a specimen from the patient;
b) performing a mitochondrial profile on the cell or specimen;
c) fixing the cell or specimen;
d) determining one or more clinical factors of the patient, and
e) classifying the patient for likelihood of clinical response to one or more cancer treatments;
wherein the one or more clinical factors are selected to increase specificity and/or
sensitivity of the mitochondrial profile for association with clinical response,

2. The method of claim 1, wherein the solid tumor is selected from non-small lung cell carcinoma, ovarian cancer, melanoma, breast cancer, prostate cancer, lung cancer, pancreatic cancer, colon cancer, hepatic cancer, and brain cancer.

3. The method of claim 1, wherein the cancer treatment is one or more of anti-cancer drugs, chemotherapy, surgery, adjuvant therapy, and neoadjuvant therapy.

4. The method of claim 3, wherein the cancer treatment is one or more of a BH3 mimetic, Her2 antibody, Gemtuzitnab, cisplatinin, EGFR inhibitor, Trail-1 ligand, epigenetic modifying agent, topoisomerase inhibitor, cyclin-dependent kinase inhibitor, and kinesin-spindle protein stabilizing agent.

5. The method of claim 3, wherein the cancer treatment is a platinum-based therapeutic.

6. The method of claim 5, wherein the platinum-based therapeutic is one or more of carboplatin, cisplatin, and oxaliplatin.

7. The method of claim 3, wherein the cancer treatment is cytarabine or a cytarabine-based chemotherapy.

8. The method of claim 4, wherein the cancer treatment is a BH3 mimetic.

9. The method of claim 8, wherein the BH3 mimetic binds to one or more of BCL2, BCLXL, and MCL1.

10. The method of claim 3, wherein the cancer treatment is an inhibitor of MCL1.

11. The method of claim 1, wherein the mitochondrial profile comprises
a) permeabilizing the cancer cells;
b) determining a change in mitochondrial membrane potential upon contacting the permeabilized cells with one or more BH3 domain peptides and/or BH3 mimetic, using a fixable mitochondrial membrane potential dependent dye;
c) fixing the cells; and
d) correlating a shift in mitochondrial membrane potential with chemosensitivity of the cells or specimen to apoptosis-inducing chemotherapeutic agents.

12. The method of claim 1, wherein the mitochondrial profile comprises use of one or more BH3 domain peptides selected from the group consisting of BIM, BIM2A, BAD, BID, HRK, PUMA, NOXA, BMF, BIK, and PUMA2A.

13. The method of claim 12, wherein the one or more BH3 domain peptides are used at a concentration of 0.1 μM to 200 μM.

14. The method of claim 1, wherein the mitochondrial profile comprises using a BH3 mimetic compound that binds to all or selected members of the anti-apoptosis Bcl-2 family proteins.

15. The method of claim 14, wherein the BH3 mimetic is used at a concentration of 0.01 μM to 100 μM in permeabilized cells.

16. The method of claim 14, wherein the BH3 mimetic is used at a concentration of 0.01 μM to 100 μM in intact cells.

15. The method of claim 1, wherein the specimen is a biopsy from a frozen tumor tissue specimen, that has been cryosectioned, treated with mitochondrial membrane potential perturbing reagents, and fixed.

16. The method of claim 1, wherein the specimen is a cancer stem cell.

17. The method of claim 1, wherein the specimen is derived from a biopsy of the solid tumor.

18. The method of claim 17, wherein the specimen is derived from the biopsy of a colorectal, breast, prostate, lung, pancreatic, renal, or ovarian primary tumor.

19. The method of claim 1, wherein the specimen is of epithelial origin.

20. The method of claim 19, wherein the epithelial specimen is enriched by selection from a biopsy sample with an anti-epithelial cell adhesion molecule (EpCAM) or other epithelial cell binding antibody bound to solid matrix or bead.

21. The method of claim 1, wherein the specimen is of mesenchymal origin.

22. The method of claim 21, wherein the mesenchymal specimen is enriched by selection from a biopsy sample with a neural cell adhesion molecule (N-CAM) or neuropilin or other mesenchymal cell binding antibody bound to a solid matrix or bead.

23. The method of claim 1, wherein the clinical factor is one or more of age, cytogenetic status, performance, histological subclass, gender, and disease stage.

24. The method of claim 1, further comprising measurement of an additional biomarker selected from mutational status, single nucleotide polymorphisms, steady state protein levels, and dynamic protein levels.

25. The method of claim 1, wherein the method further comprises predicting a clinical response in the patient.

26. The method of claim 25, wherein the clinical response is at least about 1, about 2, about 3, or about 5 year progression/event-free survival.

27. The method of claim 1, wherein the likelihood of clinical response is defined by the following equation:
$$\% Priming = [ 100 * ( \frac{DMSO AUC - {Peptide}_{1} AUC}{DMSO AUC - {CCCP}_{avg} AUC} ) ] {Peptide}_{1} + [ 100 * ( \frac{DMSO AUC - {Peptide}_{2} AUC}{DMSO AUC - {CCCP}_{avg} AUC} ) ] {Peptide}_{2} + … / ( n peptides )$$
wherein:
the AUC comprises either area under the curve or signal intensity;
the DMSO comprises the baseline negative control; and
the CCCP (Carbonyl cyanide m-chlorophenyl hydrazone) comprises the positive control.

28. The method of claim 27, wherein the area under the curve is established by LI-COR.

29. The method of claim 27, wherein the area under the curve is established by microscopy readout.

30. The method of claim 1, wherein the tumor is breast cancer and/or non-small cell lung cancer and the clinical factor is an age profile and/or cytogenetic status.

31. The method of claim 12, wherein the BH3 domain peptides are selected from the group consisting of SEQ ID NOS:1-16.
